(19) United States
(12) Patent Application Publication
(10) Pub. No.: US 2006/0085051 A1
(43) Pub. Date: Apr. 20, 2006

(54) ELECTRICAL IMPLANTS

(76) Inventor: Michael H. Fritsch, Indianapolis, IN (US)

Correspondence Address: VINCENT L. CARNEY LAW OFFICE, P.O. BOX 80836, LINCOLN, NE, 68501-0836, US

(21) Appl. No.: 10/904,018

(22) Filed: Oct. 19, 2004

(51) Int. Cl. A61N 1/08

(52) U.S. Cl. 607/61

(57) ABSTRACT

An external module is mounted to a person and transmits energy in the light spectrum through the skin to an internal module which converts it to d.c. current with a film photocell. The d.c. current can be used to charge batteries for powering an implant without a break in the skin and to power an implant directly. Light signals can also be transmitted through the skin from an internal module to the external module to monitor implants, battery charging equipment, batteries and patient functions. Control signals can be transmitted from the external module to the internal module. The energy may be in the wavelength range of 1×10⁻⁴ to 1×10⁻⁹ meters and preferably in the wavelength range of 4×10⁻⁷ to 8×10⁻⁷ meters.

### BACKGROUND OF THE INVENTION

This invention relates to apparatus and methods for supplying energy to electrically operated implants.

It is known to transcutaneously supply power and control signals to electrically operated implants in animals and most commonly in humans. One type of known apparatus for supplying power to such devices transmits the power and/or control signals through the skin as electromagnetic energy to avoid breaking the skin. In some such apparatuses, the energy is stored in implanted storage batteries that supply power to battery-operated implants.

In some prior art systems of this type, alternating current from an external source is induced in an implanted receiving coil and conducted to the storage battery or batteries or transmitted directly to the electrically operated implant. Prior art systems of this type are disclosed in U.S. Pat. Nos. 6,525,512; 6,227,204; 6,073,050 and 5,411,537.

This prior art type of apparatus and methods for supplying power and control signals has several disadvantages such as for example: (1) they may induce currents unintentionally in metallic parts of other implants or trigger other biological responses; and (2) they may receive interference signals on the receiving coil that disrupt control of or overload circuitry.

### SUMMARY OF THE INVENTION

Accordingly, it is an object of the invention to provide a novel implant.

It is a further object of the invention to provide a novel method for transcutaneous delivery of power to an implant.

It is a still further object of the invention to provide a novel apparatus for supplying power to an implant.

It is a still further object of the invention to provide a novel method and apparatus for wireless transfer of power to an implant.

It is a still further object of the invention to provide a novel method and apparatus for charging batteries.

It is a still further object of the invention to provide a novel method and apparatus for charging implanted batteries.

It is a still further object of the invention to provide a novel apparatus and method for transmitting energy at a wavelength that does not affect implants other than the intended implant.

It is a still further object of the invention to provide a novel apparatus and method for transmitting energy at a wavelength that does not affect biological electro-chemical functions in the human body.

It is a still further object of the invention to provide a novel apparatus and method for transmitting signals through the unbroken skin.

It is a still further object of the invention to provide a novel flexible implant.

It is a still further object of the invention to provide a flexible implantable photocell for receiving energy transmitted through unbroken skin.

It is a still further object of the invention to provide a thin, flexible implantable photocell having an area for receiving energy of at least 5 square millimeters and a thickness no greater than 1 centimeter.

In accordance with the above and other objects of the invention, energy is radiated through the unbroken skin to an implanted transducer that converts it to non-radiant electrical energy. In one embodiment, the energy is stored in batteries for powering implanted electrical apparatuses, but it may be directly applied to an implant. In the preferred embodiment, the radiant energy is electromagnetic energy at frequencies high enough to be substantially straight line in transmission and attenuated quickly so that there is no substantial difficulty in avoiding interference with biological processes, such as the rhythm of the heart, nor of implanted devices, such as pacemakers. Preferably, the transducer is photovoltaic and the electromagnetic energy is in the light wavelength range. Feedback signals may be provided such as for example by light emitting devices, such as LEDs or fluorescent devices or by converting the signals to low intensity a.c. signals for transmission through the skin, to provide data such as the intensity of the radiation that is contacting the photovoltaic device or to indicate the state of charge of the batteries or the condition of the implant or the like.

Generally, the electromagnetic energy is transmitted at a wavelength in the range of 1×10⁻⁴ to 1×10⁻⁹ meters through the skin of a patient having an implant to a photocell whereby the radiation is converted to d.c. electrical current within the patient without the need for an opening in the skin of the patient. Preferably, the electromagnetic radiation is in a wavelength range that falls within the range of 4×10⁻⁷ to 8×10⁻⁷ meters. The current can be applied to a rechargeable battery or be modulated to provide control signals to an internal transducer such as an LED for sending signals in the form of light or to an antenna for transmitting low frequency electromagnetic signals through the skin. The battery may provide power to an implant.

Signals may be transmitted through the skin from inside the patient to an external apparatus without a break in the skin using wavelengths within the same general range of wavelengths of electromagnetic energy, but preferably spaced from the range used for transmitting energy into the body to avoid interference between the two.

One feature of the invention uses the signals transmitted through the skin from an internal light emitter to control the intensity of light transmitted from an external apparatus through the skin. In one version of this embodiment, fluorescent light generated from the energy transmitted from the external apparatus is transmitted from the internal transducer to the external apparatus providing indications of the intensity of the light received by the internal transducer. The current generated by the photovoltaic cell that powers the internal apparatus, or by a separate photovoltaic cell may be applied to an LED or converted to a sufficiently high electromagnetic frequency and transmitted through the skin. Moreover, light may be generated by either the internal or external apparatus and modulated to provide information through the skin to trigger operations by an implant from outside the body or to indicate to an external apparatus or person the battery condition of storage batteries in the internal transducer.

From the above description, it can be understood that the method and apparatus for supplying power to implants of this invention has several advantages: (1) it transmits energy through the skin without an opening in the skin with no substantial risk of interference with other electrically operated implants or biological processes; (2) it is not subject to misfiring or damage from external electromagnetic signals such as emanate from electric motors, radio transmitters, power lines and the like; and (3) it is sufficiently thin and flexible to permit ready implantation in patients.

### BRIEF DESCRIPTION OF THE DRAWINGS

The above noted and other features of the invention will be better understood from the following detailed description when considered with reference to the accompanying drawings, in which:

FIG. 1 is a block diagram of an apparatus for the transcutaneous transmission of energy for powering an electrically-operated implant in accordance with an embodiment of the invention;

FIG. 2 is a simplified block diagram of an external source of power and signals used in the embodiment of FIG. 1;

FIG. 3 is a block diagram of an implanted photovoltaic unit used in the embodiment of FIG. 1 for receiving power and signals from an external source of power and signals in accordance with the embodiment of FIG. 1;

FIG. 4 is a block diagram of a power control circuit in accordance with the embodiment of FIG. 1;

FIG. 5 is block diagram of a rechargeable battery circuit useful in the embodiment of FIG. 1;

FIG. 6 is a block diagram of a programmable control system usable in the embodiment of FIG. 2;

FIG. 7 is a block diagram of another programmable control system usable in the embodiment of FIG. 2;

FIG. 8 is a block diagram of a portion of an embodiment of feedback system from an internal implanted unit to the external system of FIG. 2;

FIG. 9 is a block diagram of another portion of a feedback system from an internal unit to an external unit useable in the embodiment of FIG. 3;

FIG. 10 is a block diagram of a portion of another feedback system usable in the embodiment of FIG. 3; and

FIG. 11 is a block diagram of another embodiment of feedback system usable in the embodiment of FIG. 3.

### DETAILED DESCRIPTION

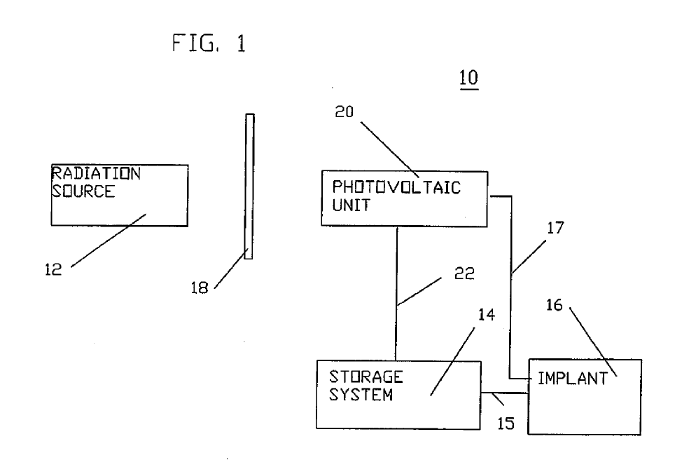

In FIG. 1, there is shown a block diagram of apparatus 10 for transcutaneously transmitting energy through the tissue 18 of a patient to an implant 16, which apparatus 10 includes a radiation source 12, a photovoltaic unit 20 and an energy storage unit 14. As shown in FIG. 1, the radiation source 12 transmits energy through the unbroken skin or deeper tissues 18 to the photovoltaic unit 20, which generates current in response to the radiation and transmits it through a shielded conductor 22 to the storage system 14. The storage system 14 stores energy for application to the implant 16 and transmits signals back to the photovoltaic unit 20 over one or more conductors 22. The implant 16 receives energy and control signals over one or more conductors 15 and transmits signals relating to its condition over conductor 17.

While many photovoltaic systems are available including photodiode arrays of several types, flexible thin film photovoltaic systems are preferred. They should be flexible enough for insertion in the cavity prepared by the surgeon and may be used for subcutaneous use wherever it is implanted including intra-abdominal, intra-cranial or intra-thoracic implantation. One such system is sold by Big Frog Mountain, 100 Cherokee Boulevard Suite 321, Chattanooga, Tenn. 37405, USA under the trademark PowerFilm. The photovoltaic systems should be encased in a light-passing tissue-compatible material such as silicon. In this specification, the words apparatus, apparatuses, implant or photovoltaic unit means one or more functional units which may be separate or enclosed in one or more housings.

With this apparatus, radiant energy such as visible light can be used to transmit power and signals to and from internally implanted units. Thus, batteries for an implanted device such as a cochlear implant, heart monitoring or control devices or a medication pump can be recharged or power sent directly to the implant, or control signals and monitoring signals can be sent back to an external apparatus. Because very short wavelengths of radiant energy are used, the signals can be isolated to avoid interference.

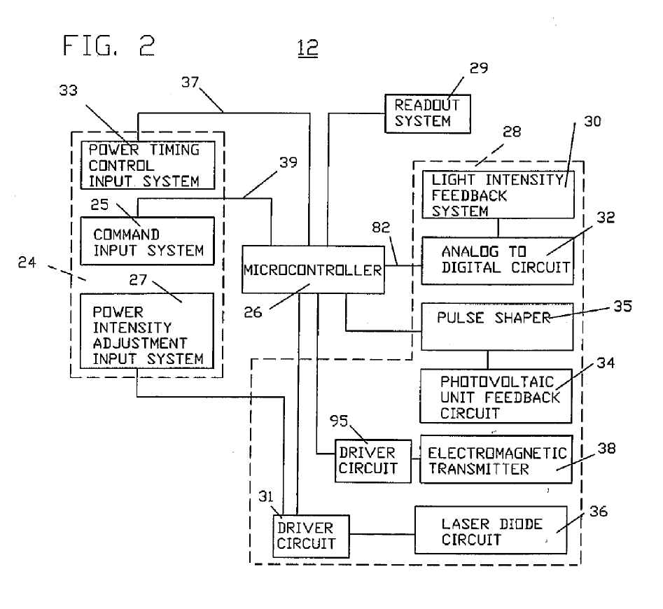

In FIG. 2, there is shown a block diagram of one embodiment of a radiation source 12 having an input control section shown generally at 24, a microcontroller 26, a readout system 29 and a transmission system 28. The input control section 24 communicates with the microcontroller 26 and the transmission system 28 to control the power and signals transmitted transcutaneously to the photovoltaic unit 20 (FIG. 1). To aid in this process, the microcontroller 26, in addition to receiving some signals from the input control section 24 and having data stored in its memory, also receives signals from the transmission system 28. With these signals and stored information, the microcontroller 26 transmits signals to provide to the readout unit 29 a readout of conditions that are internal to the person and to generate control signals based on conditions that are internal to the person having the implant for use by the transmission system 28.

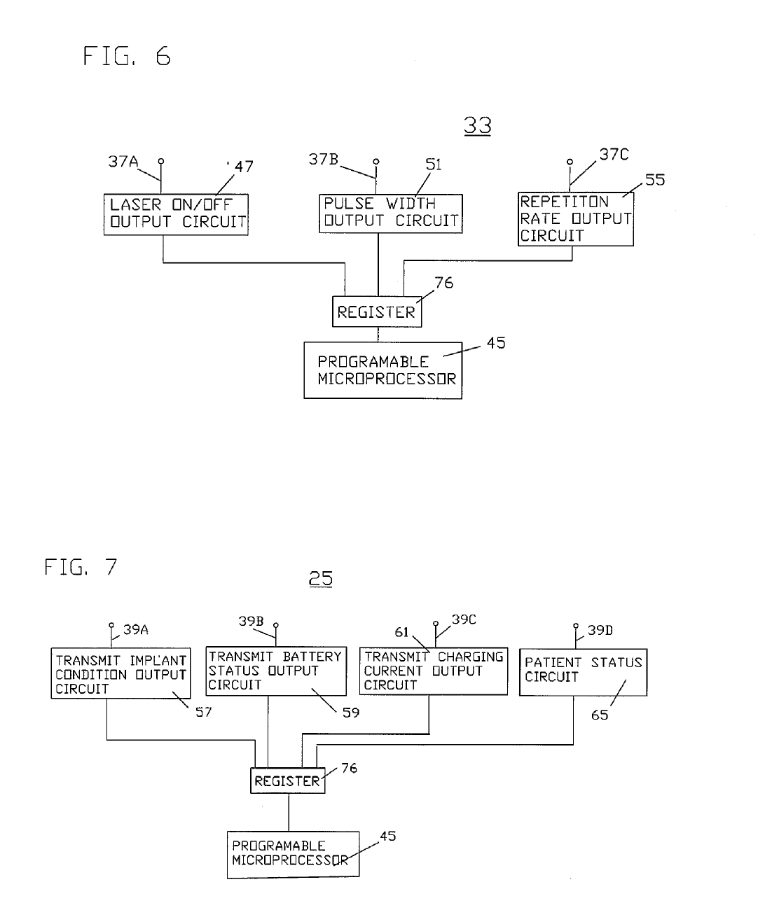

The input control section 24 includes a power timing control input system 33, a command input system 25 and a power intensity adjustment input system 27. The power timing control input system 33 communicates with the microcontroller 26 through conductors 37A-37C (FIG. 6) indicated as 37 in FIG. 2 and the command input system 25 communicates with the microcontroller 26 through conductors 39A-39D (FIG. 7) indicated as 39 in FIG. 2 to supply power control signals and command signals to the microcontroller 26 for use in controlling the time and pulse transmission of power to and initiating and terminating operations in the photovoltaic unit 20 (FIG. 1) respectively.

The power control signals control the application of power to supply energy to the implant 16 (FIG. 1) or storage system 14 (FIG. 1) and the command signals which may be used for several control purposes such as for example to trigger a readout of signals from the photovoltaic unit 20 indicating the condition of the storage system 14 or implant 16 (FIG. 1). In response to the power control signals from the power timing control input system 33, the microcontroller 26 controls the transmission system 28 that transmits radiant energy to the photovoltaic unit 20 (FIG. 1). Similarly, in response to the command signals, the microcontroller 26 controls the transmission system 28 that supplies command control signals to the photovoltaic unit 20 (FIG. 1). The power intensity adjustment input system 27 communicates with the transmission system 28 to adjust the amount of power by controlling the radiation intensity that is generated by the transmission system 28 for transmission to the photovoltaic unit 20 (FIG. 1).

The transmission system 28 includes the driver circuits 31 and 95, a light intensity feedback system 30, an analog-to-digital converter circuit 32, a pulse shaper 35, a photovoltaic unit feedback circuit 34 and a laser diode circuit 36. With this arrangement, the laser diode circuit 36 irradiates the photovoltaic unit 20 (FIG. 1) through tissue 18 (FIG. 1) to generate current for charging the storage system 14 (FIG. 1) and for providing control signals. In one embodiment, the intensity of the radiation is controlled by the driver circuit 31 by adjusting the power in response to signals received from the light intensity feedback system 30. In this embodiment, the light intensity feedback system 30 receives a signal from the photovoltaic unit 20 (FIG. 1) and transmits the signal to the analog-to-digital converter circuit 32 which transmits it to the microcontroller 26 through a conductor indicated at 82. The microcontroller 26 compares the signal from the analog-to-digital converter 32 and the signal from the power timing control input system 33 to control the power to the laser diode circuit 36 by controlling the amplification from the driver circuit 31. While a laser diode circuit 36 is used in the specific embodiment of FIG. 2, other types of radiators may be used and a wide range of wavelengths of the electromagnetic spectrum may be used.

In this embodiment, signals from the light intensity feedback system 30 and analog-to-digital circuit 32 automatically control the amplification of the driver circuit 31 through the microcontroller 26 to which they are connected. This control automatically limits the power transferred to the internal unit by the laser diode circuit 36 to a preset safe value while permitting the surgeon to set the intensity, the pulse width and the repetition rate of the pulses of light from the laser diode so that the intensity is high enough to penetrate the tissue 18 (FIG. 1) but the repetition rate and the pulse width are sufficient to generate an adequate charging current but provide low enough power to prevent harm. The photovoltaic unit feedback circuit 34 senses signals from the photovoltaic unit 20 (FIG. 1) indicating the state of charge of the storage system 14 (FIG. 1). In another embodiment, an operator adjusts the power intensity adjustment input system 27 until the analog-to-digital circuit 32 is receiving fluorescent light, LED or other electromagnetic energy and emitting a signal in response thereto but the light intensity feedback system 30 is not receiving sufficient light to provide a signal. Information concerning both the conditions internal to the patient and the settings of the external apparatus can be indicated on the readout system 29. The fluorescent light from the external unit and the fluorescent light emitted by the internal unit in response to the light from the external unit are preferably of different wavelengths.

In response to signals from the microcontroller 26, the driver circuit 95 supplies command signals to the electromagnetic transmitter 38 which sends signals transcutaneously to a photovoltaic unit 20 (FIG. 1). These signals are weak and do not cause difficulties with other equipment since they only need to be received after traveling a short distance and do not need to transmit substantial power. The power needs are supplied by the laser diode circuit 36 which avoids disrupting other electrical equipment or biological functions because it is light energy rather than the lower frequency energy and is thus attenuated quickly and transmitted along substantially straight line paths. Although low-frequency low-amplitude electromagnetic signals, for example radio frequency or lower frequencies are used to transmit command signals in the embodiment of FIG. 2, light signals formed by modulating the laser diode in the laser diode circuit 36 or by a separate light path to a separate photocell from the one receiving the energy to charge the batteries could be used. To receive information from the implant 16 (FIG. 1) concerning the condition of the implant and batteries, the photovoltaic unit feedback circuit 34 receives pulses and transmits them though pulse shaper 35 to the microcontroller 26.

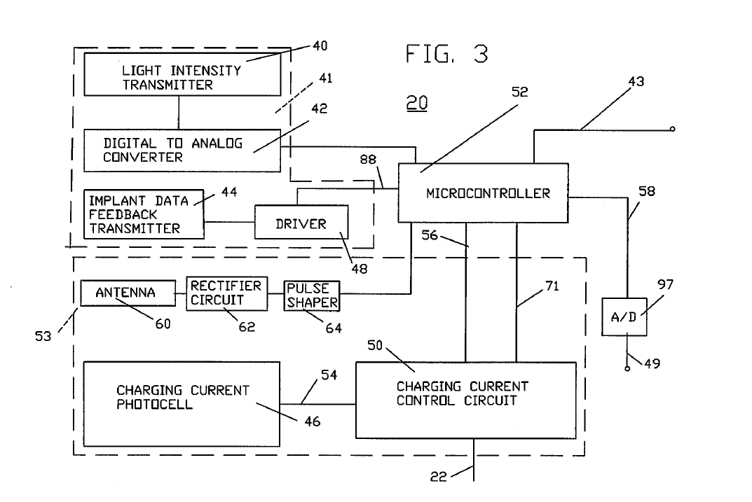

In FIG. 3, there is shown a block diagram of the photovoltaic unit 20 having a feedback radiation system 41, a charging system analog-to-digital converter 97 for the charging system, a microcontroller 52 and a charging current generation and control circuit 53. The feedback radiation system 41 is connected to the microcontroller 52 to transmit information transcutaneously to the external apparatus concerning light intensity and the condition of internal apparatus components using radiant energy. The charging current generation and control circuit 53 receives both signals and energy for charging batteries and powering implants from the external apparatus and supplies power to the batteries or implants and signals to the microcontroller 52. A conductor 43 provides signals from the microcontroller 52 to the implant 16 (FIG. 1), and the analog-to-digital converter 97 receives signals from the storage system 14 (FIG. 1) on conductor 49, converts them to digital form and conducts them to the microcontroller 52.

The charging-current generation-and-control circuit 53 includes a charging current photocell 46, a charging-current control circuit 50, an antenna 60, a rectifier circuit 62 and a pulse shaper 64. Current from the charging current photocell 46 is controlled by the charging current control circuit 50 which transmits it to the storage system 14 (FIG. 1) through a conductor 22 at a preset voltage when the batteries are not fully charged and transmits signals to the microcontroller 52 through a conductor 71 indicating the amount of current being generated. It transmits signals that control the charging current to maintain it at a rate that does not cause gas formation or overheating of the battery or batteries. The batteries stop receiving current when fully charged. The antenna 60 receives command signals from the external apparatus at a lower frequency than light and transmits them to the rectifier circuit 62 or other suitable circuitry. The rectifier circuit 62 is connected to the pulse shaper 64 which forms pulses of the proper amplitude and transmits them to the microcontroller 52 for use in controlling other operations as programmed in the command input system 25 (FIG. 2).

For these functions, the charging current generation and control circuit 53 receives energy: (1) radiated from the laser diode circuit 36 (FIG. 2) that is in the external apparatus and converts it to energy used by the internal transducer; and (2) radiated from the electromagnetic transmitter 38 (FIG. 2) in the external apparatus and conducts it to the microcontroller 52 to provide control signals to the internal transducer. More specifically in the preferred embodiment, the charging current generation and control circuit 53 converts radiant light energy to d.c. current for charging batteries or for directly powering one or more implants and converts radiant energy of a lower frequency or modulated light energy to control signals for application to the microcontroller 52.

In the preferred embodiment, the charging current photocell 46 is a flexible unit that can be installed conveniently in the patient and be bent as needed to conform to the requirements of the cavity into which the surgeon chooses to implant it. In one embodiment, the photocell 46 is a film-like implantable photocell formed of sheet-like material selected by the surgeon for thickness and flexibility to fit within the patient's body at the selected location. One such flexible thin film photovoltaic system sold by Big Frog Mountain, 100 Cherokee Boulevard Suite 321, Chattanooga, Tenn. 37405, USA under the trademark PowerFilm is preferred. The photovoltaic systems should be encased in a light-passing tissue-compatible material such as silicone.

To provide control signals to the radiation source 12, (FIG. 1), the microcontroller 52 is electrically connected to the storage system 14 (FIG. 1) through the analog-to-digital converter 97 to receive digital signals indicating the battery voltage from conductor 58. The digital-to-analog converter 42 is electrically connected to the storage system 14 (FIG. 1) through conductor 49. With this arrangement, the microcontroller 52 receives signals indicating the condition of the battery or batteries so as to terminate charging before an over-charge condition exists and to provide warnings and control if the voltage falls to an unsafe or undesirable level. The microcontroller 52 provides signals on conductor 56 to control the flow of current to the storage system 14 (FIG. 1) on conductor 22 and from the charging current photocell 46. It is also able to communicate the battery condition or other information by controlling pulses from an implant data feedback transmitter 44 by controlling a driver 48.

The feedback radiation system 41 includes a light intensity transmitter 40, a digital-to-analog converter 42, an implant data feed back transmitter 44 and a driver 48 for the feedback data transmitter. The feedback radiation system 41 transmits energy containing information from the internal transducer back to the external apparatus. In one embodiment, instead of a light intensity transmitter 40, a low frequency electromagnetic transmitter is used. In other embodiments, it is a fluorescent system or an LED system, a laser system or other light emitting systems. In the preferred embodiment, the function of the feedback radiation system 41 is to control the intensity of at least one type of radiation from the external apparatus but in other embodiments can provide information to the microcontroller 26 (FIG. 2) about the status or operating condition of the internal apparatus 41.

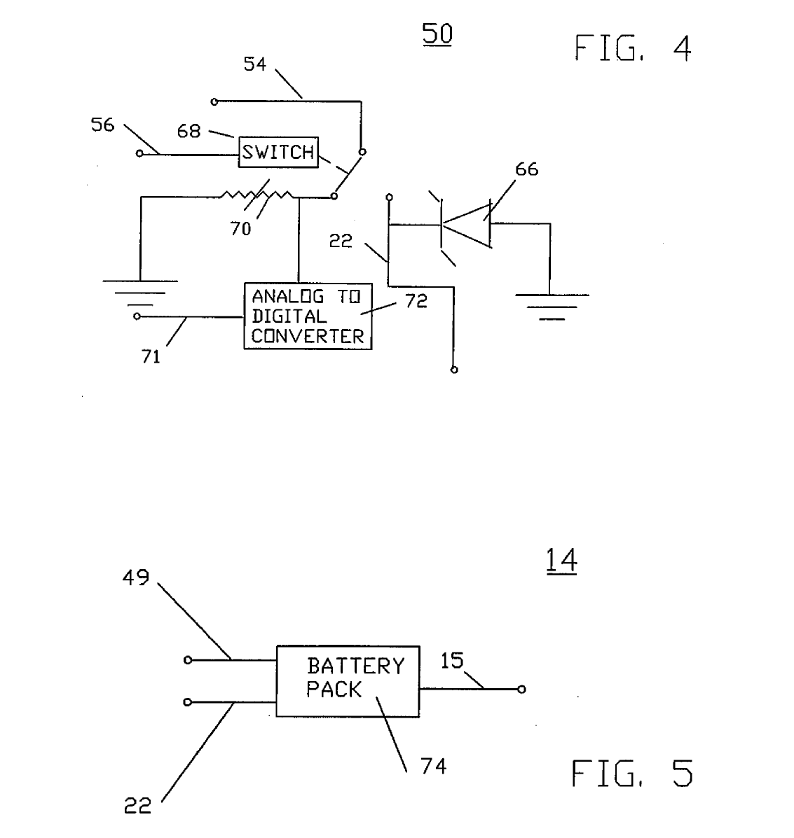

In FIG. 4, there is shown a simplified schematic diagram of the charging current control circuit 50 having a single-pole double-throw switch 68, a voltage-control Zener diode 66 has its anode grounded and its cathode connected to one contact of the single-pole double-throw switch conductor 22 to hold the voltage at a fixed amount for charging the batteries. The variable resistor 70 is connected between the conductor 54 and ground to receive the charging current when the switch 68 is closed to the analog-to-digital converter 72 to obtain a current reading and open circuited to the batteries. At this time, the analog-to-digital converter 72 is connected to receive the voltage drop across the variable resistor 70 and thus transmits a current reading to the microcontroller 52 (FIG. 3) through conductor 71. The switch 68 is opened to the variable resistor 70 and analog-to-digital converter 72 and closed to conductor 22 when battery voltage is low by a signal from the microcontroller 52 (FIG. 3) on conductor 56 to permit current to flow from the charging current photocell 46 (FIG. 3) through conductor 54 to conductor 22 and from there to the storage system 14 (FIG. 1). When the batteries are fully charged, the switch 68 is opened to conductor 22 and closed to the variable resistor 70 and analog to digital converter 72. At this time, the charging current being monitored is checked to be sure it is within the requirements for the batteries or implant and if not, the power from the laser diode circuit 36 (FIG. 2) is adjusted. When it is within specifications, the laser is terminated and the readout system 29 (FIG. 2) indicates that the external unit can be disconnected.

In FIG. 5, there is shown a block diagram of the storage system 14 having a rechargeable battery pack 74 connected to the conductor 22 to receive current during charging and connected to conductor 15 to supply power to the implant 16 (FIG. 1). The conductor 49 is connected to supply a signal indicating the voltage state of the battery pack 74 to the microcontroller 52 (FIG. 3) through the analog-to-digital converter 97 (FIG. 3) to be used in determining when to close switch 68 (FIG. 4) to conductor 22 to supply current to the battery pack 74.

In FIG. 6, there is shown a block diagram of the power timing control input system 33 having a programmable microprocessor 45 with a keyboard, a register 76, a laser on-off output circuit 47, a laser pulse width output circuit 51, a laser repetition rate output circuit 55 and conductors 37A-37C. The microprocessor 45 is connected to the register 76 and programmed to cause the register 76 to select conductors and supply a signal to them for application to the microcontroller 26 (FIG. 2) through conductors 37A-37C according to the pulse shaping and amplitude control in one of the output circuits 47, 51 or 55. The laser on/off output circuit 47 is connected to the microcontroller 26 (FIG. 2) through conductor 37A to supply a signal controlling the time the laser diode circuit 36 (FIG. 2) is turned on and off; the pulse width output circuit 51 is connected to the microcontroller 26 (FIG. 2) through conductor 37B to supply a signal controlling the pulse width of the light from the laser diode circuit 36 (FIG. 2) which affects the amount of current generated and the power transferred to the batteries; the repetition rate output circuit 55 is connected to the microcontroller 26 (FIG. 2) through conductor 37C to supply a signal controlling the repetition rate of pulses from the laser diode circuit 36 (FIG. 2), which together with the pulse-width and intensity, controls the power delivered to the photovoltaic unit 20 (FIG. 1).

With this circuit, an entry into the keyboard of the programming computer 45 provides a signal to the microcontroller 26 (FIG. 2): (1) through conductor 37A from the laser on-off output circuit 47 indicating the time duration over which power is to be applied; (2) a signal through conductor 37B from the pulse width output circuit 51 to control the length of time the laser is energized in each cycle (pulse width of the laser); and (3) a signal through conductor 37C from the repetition rate output circuit 55 to control the time duration of a cycle and the frequency of each cycle. These values determine the amount of time the power is applied and the time of the pulses in a manner to balance energy need with heat dissipation when the intensity of the laser beam is set by the power intensity adjustment input system 27 (FIG. 2).

In FIG. 7, there is shown a block diagram of the command input system 25 having the programmable microprocessor 45, the register 76, a transmit implant condition output circuit 57, a transmit battery status output circuit 59, a transmit charging current output circuit 61 and a patient status circuit 65. The programmable microprocessor with keyboard 45 permits the operator to enter a value and have the register 76 to which it is connected register a count that energizes a selected circuit such as the transmit implant condition output circuit 57, the transmit battery status output circuit 59, or the transmit charging current output circuit 61 or the patient status circuit 65. Each of these circuits is connected to the microcontroller 26 (FIG. 2) through a different one of the conductors 39A-39D which in turn is connected to the driver circuit 95 (FIG. 2) to cause the electromagnetic transmitter 38 (FIG. 2) to transmit commands to the internal apparatus to initiate a readout from the internal apparatus to the external apparatus of the implant condition, battery status, charging current value or patient status. With this arrangement, command signals can be transmitted to the internal unit, causing the internal implant conditions to be transmitted back to the external unit for use in controlling the transmission system 28 (FIG. 2) and for display in the readout system 29 (FIG. 2).

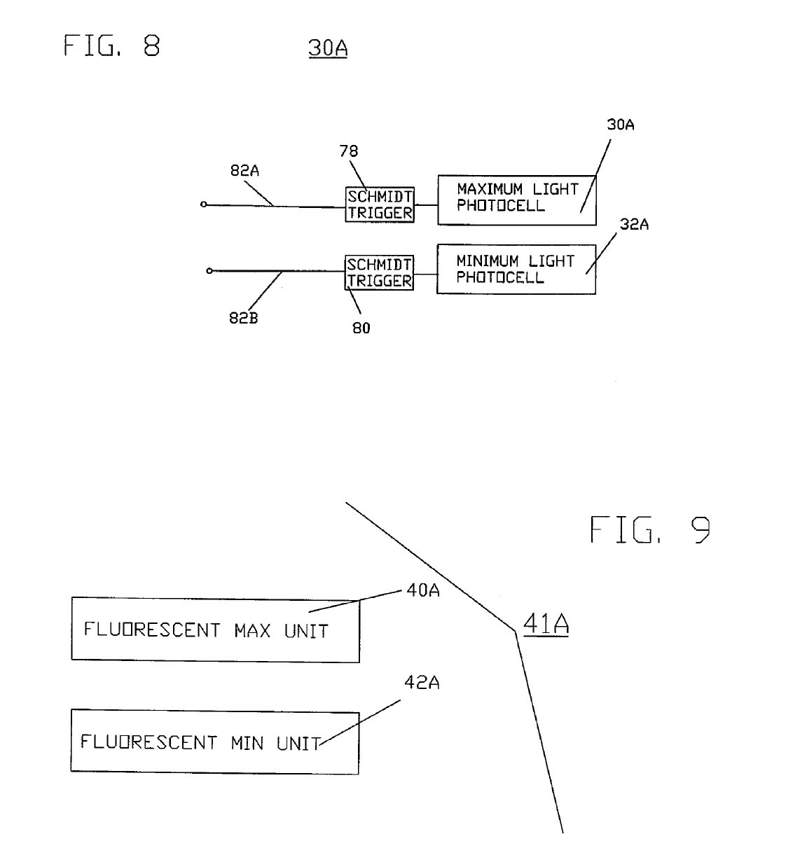

In FIG. 8, there is shown a block diagram of one embodiment of a light intensity feedback system 30A, which may be used in the embodiment of FIG. 2 instead of the light intensity feedback system 30. The light intensity feedback system 30A has maximum and minimum light photocells 30A and 32A. In this embodiment, signals from the maximum and minimum light photocells 30A and 32A are applied to the microcontroller 26 (FIG. 2) through Schmidt triggers 78 and 80 and conductors 82A and 82B respectively. The intensity of the light emitted by the laser diode 36 (FIG. 2) is controlled by the light received from the fluorescent unit, LED or other light emitted in the light intensity transmitter 40 (FIG. 3) by the maximum light photocell 30A and from the fluorescent unit, LED or other light emitter by the minimum light photocell 32A rather than by lower frequency electromagnetic radiation transmitted by an antenna in the interior apparatus.

In FIG. 9, there is shown a block diagram 41A of a portion of the one embodiment of the photovoltaic unit 20 that may cooperate with the embodiment of light intensity feedback system 30A (FIG. 8) having a fluorescent maximum light-mode, feedback-signal unit 40A and a fluorescent minimum light-mode, feedback-signal unit 42A or LED or other light emitter or electromagnetic emitter for transmitting signals indicating the intensity of the light transmitted through the skin of the patient.

In this embodiment, light from the laser diode 36 (FIG. 2) impinges upon and activates the fluorescent maximum and minimum light intensity units 40A and 42A and the charging current photocell 46 (FIG. 3).

Each of these units 40A and 42A is sealed in a light passing seal but the fluorescent maximum light intensity unit 40A is colored to filter out some of the light so that it does not fluoresce with light of low intensity but does fluoresce with light above an intensity that causes excessive heating or discomfort of the patient. The power to the laser diode 36 (FIG. 2) is set either manually by the microcontroller 26 (FIG. 2) to cause the minimum light photocell 32A (FIG. 8) positioned next to but on the external side of the tissue 18 (FIG. 1) to receive fluorescent light from the implanted fluorescent minimum unit 42A while the maximum light photocell 30A (FIG. 8) does not receive light from the implanted fluorescent maximum unit 40A. This causes the Schmidt trigger 80 (FIG. 8) to fire but not the Schmidt trigger 78 (FIG. 8) to apply a signal to the microcontroller 26 (FIG. 2) through conductor 82B (indicated as one of the conductors 82 in FIG. 2) but not through conductor 82A. On the other hand, if the light transmitted from the laser diode circuit 36 (FIG. 2) is too intense, the microcontroller 26 (FIG. 2) receives signals on both conductors 82A and 82B (FIG. 8) causing the microcontroller 26 to reduce the width of the pulses and the repetition rate.

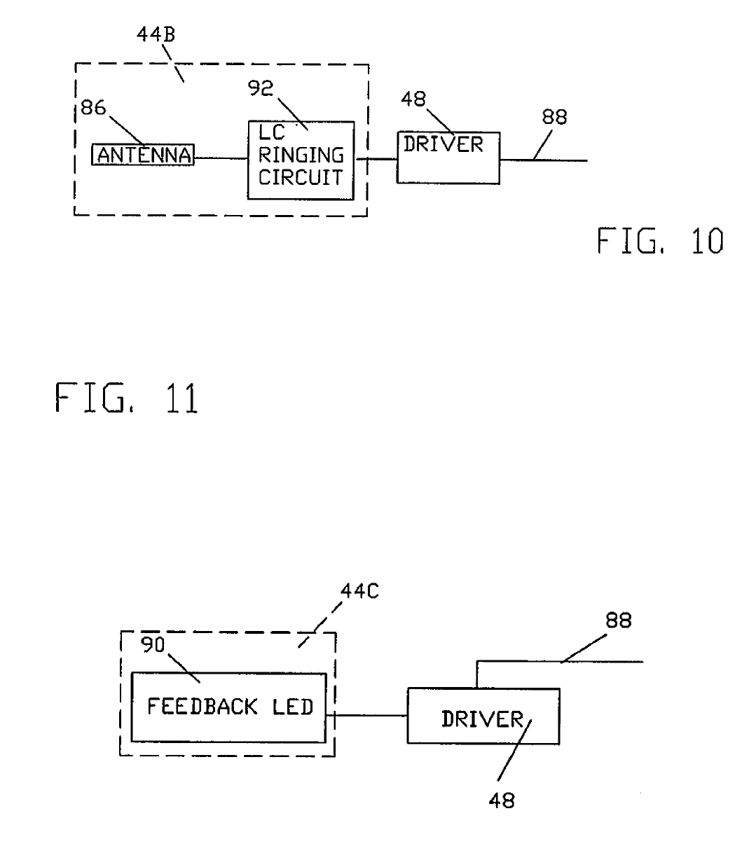

In FIG. 10, there is shown a block diagram of another embodiment of implant data feedback transmitter 44B for transmitting signals to an antenna type light intensity feed back system 30 (FIG. 2) having an LC ringing circuit 92, a driver 48 and an antenna 86. The driver 48 is electrically connected to the microcontroller 52 (FIG. 3) through conductor 88 to receive pulses indicating the data requested by the command input system 25 (FIG. 2). The driver 48 amplifies the pulses from the microprocessor 52 (FIG. 3) and applies them to the LC ringing circuit 92 which responds by generating oscillations for each pulse from the driver 48 and applying them to the antenna 86 for transcutaneous transmission to the photovoltaic unit feedback circuit 34 (FIG. 2) for transmission to the microcontroller 26 (FIG. 2) through the pulse shaper 35 (FIG. 2). The LC ringing circuit 92 is a ringing resonant circuit that oscillates in response to the pulse from the driver 48.

In FIG. 11, there is shown another embodiment of implant data feedback transmitter 44C having a feedback LED 90 connected to the driver 48 to receive pulses on conductor 88 from the microcontroller 52 (FIG. 3) indicating implant data. In this embodiment, the photovoltaic unit feedback circuit 34 (FIG. 2) includes a photocell that receives light pulses transmitted by the LED which is located adjacent to the LED 90. With these connections, the feedback LED 90 transmits light transcutaneously to a photocell in the photovoltaic unit feedback circuit 34 to provide the information to the microcontroller 26 (FIG. 2).

In operation, energy is radiated through the unbroken skin 18 (FIG. 1) by radiant energy to an implanted transducer which in the preferred embodiment is a photovoltaic unit 20. The photovoltaic unit 20 converts the radiant energy to non-radiant electrical energy, which in the preferred embodiment is in the form of d.c. current. The energy is stored in batteries which in the preferred embodiment are the battery pack 74 (FIG. 5) that supplies power and control signals to the implant 16 (FIG. 1). In the preferred embodiment, the radiant energy is electromagnetic energy at frequencies high enough to be a substantially straight line in transmission and attenuated quickly so that there is no substantial difficulty in avoiding: (1) interference with biological processes such as the rhythm of the heart by the energy transmitted into the body of a patient; (2) interference with implanted devices such as pacemakers; nor (3) interference with signals from externally generated electromagnetic noise such as that generated by electrical motors or by broadcast stations. Preferably, the transducer is photovoltaic and the electromagnetic energy is in the light wavelength range. Feedback signals are provided by light emitting devices such as photodiodes to indicate the state of charge.

Generally, the electromagnetic energy is transmitted at a wavelength in the range of 1×10⁻⁴ to 1×10 meters through the skin of a patient to a photocell whereby the light is converted to current within the patient without a break in the skin of the patient. The current can be applied to a rechargeable battery or be modulated to provide control signals to an internal transducer. The battery may provide power to an implant. Preferably, the electromagnetic radiation is in a wavelength range of 4×10⁻⁷ to 8×10⁻⁷. Signals may be transmitted through the skin from inside the patient to an external apparatus without a break in the skin using the same general range of wavelengths of electromagnetic energy.

In one embodiment, the intensity of light transmitted from an external apparatus such as the radiation source 12 (FIG. 1) through the skin illustrated at 18 (FIG. 1) to supply power for an implant and/or signals to control an implant is indicated and controlled by signals from a light generator within the internal transducer. In one version of such an embodiment, fluorescent light generated from the energy transmitted from the external apparatus or radiation source 12 (FIG. 1) causes fluorescence in one or more fluorescent units such as 40 and 42 although more than two may be used. The fluorescent units are each coated with a different amount of radiation filtering material so the radiation from the external apparatus causes fluorescence in one or more of the fluorescent units but not in all of them. Thus, the intensity of the radiation from the external apparatus is indicated by the amount of filtering material that attenuates the radiation sufficiently to prevent fluorescence that can be detected through the skin. The location of the fluorescent units that are fluorescing indicates the strength of radiation from the external apparatus that is penetrating the skin. The transmission of energy for the storage system 14 (FIG. 1) is controlled by a switch 68 (FIG. 4) which in turn is controlled by a microcontroller that receives signals from the storage system and controls feedback signals through the implant data feedback transmitter 44 (FIG. 3) and the application of power from the charging current photocell 46 (FIG. 3) through the charging current control circuit 50 (FIG. 3).

From the above description, it can be understood that the method and apparatus for supplying power to implants of this invention has several advantages, such as for example: (1) it transmits energy through the skin without an opening in the skin with no substantial risk of interference with other electrically operated implants or biological processes; (2) it is not subject to misfiring or damage from external electromagnetic signals such as emanate from electric motors, radio transmitters, power lines and the like; and (3) it is sufficiently thin and flexible to permit ready implantation in patients.

While a preferred embodiment of the invention has been described with some particularity, many modifications and variations of the preferred embodiment are possible in the light of the above teachings. Accordingly, it is to be understood that, within the scope of the appended claims, the invention may be practiced other than as specifically described.

What is claimed is:

1. A method of supplying energy to an implant comprising the steps of:
transmitting electromagnetic energy having a wavelength in the range of 1×10⁻⁴ to 1×10⁻⁹ meters through skin of a patient to a photocell whereby light is converted to current within the patient without a break in the skin of the patient;
applying the current to a rechargeable battery; and
applying energy from the battery to an implant.

2. A method in accordance with claim 1 in which the electromagnetic energy is in a wavelength range of 4×10⁻⁷ to 8×10⁻⁷.

3. A method in accordance with claim 1 further including the step of transmitting signals through the skin from inside the patient to an external apparatus without a break in the skin.

4. A method in accordance with claim 3 further including the step of using the signals transmitted through the skin to control the intensity of light transmitted from the external apparatus through the skin to an internal transducer.

5. A method in accordance with claim 3 further including the step of using the signals transmitted through the skin to indicate the battery condition of storage batteries in an internal transducer.

6. A method in accordance with claim 1 further including the steps of:
modulating the electromagnetic energy transmitted through the skin of the patient; and
using the modulated energy to transmit signals to an internal transducer.

7. A method in accordance with claim 3 further including the step of using the signals transmitted through the skin to indicate the patient's condition.

8. A method of supplying energy to an implant comprising the steps of:
transmitting electromagnetic energy having a wavelength in the range of 1×10⁻⁴ to 1×10⁻⁹ meters through skin of a patient to a photocell whereby light is converted to current within the patient without a break in the skin of the patient;
applying the current to the implant.

9. A method in accordance with claim 8 in which the current supplies power to the implant used in the operation of the implant.

10. A method in accordance with claim 8 further including the steps of:
modulating the electromagnetic energy transmitted through the skin of the patient; and
using the modulated energy to control the operation of the implant.

11. Apparatus for supplying energy to an implant comprising:
a source of electromagnetic energy;
means for transmitting at least a portion of the electromagnetic energy having a wavelength in the range of 1×10⁻⁴ to 1×10⁻⁹ meters through skin of a patient to a photocell whereby light is converted to current within the patient without a break in the skin of the patient;
first conductor means connected between the photocell and a rechargeable battery whereby current is conducted to the rechargeable battery from the photocell; and
second conductor means connected between the rechargeable battery and the implant whereby current is conducted from the rechargeable battery to the implant.

12. An apparatus in accordance with claim 11 in which the electromagnetic energy is in a wavelength range of 4×10⁻⁷ to 8×10⁻⁷.

13. An apparatus in accordance with claim 11 further comprising means for transmitting signals through the skin from inside the patient to an external apparatus without a break in the skin.

14. An apparatus in accordance with claim 13 further comprising means for using the signals transmitted through the skin to control the intensity of light transmitted from the external apparatus through the skin to an internal transducer.

15. An apparatus in accordance with claim 13 further comprising means for using the signals transmitted through the skin to indicate the battery condition of storage batteries in an internal transducer.

16. An apparatus in accordance with claim 11 further comprising:
means for modulating the electromagnetic energy transmitted through the skin of the patient; and
means for using the modulated energy to transmit signals to an internal transducer.

17. An apparatus in accordance with claim 13 further including the step of using the signals transmitted through the skin to indicate the patient's condition.

18. Apparatus for supplying energy to an implant comprising:
a source of electromagnetic energy;
means for transmitting at least a portion of the electromagnetic energy having a wavelength in the range of 1×10⁻⁴ to 1×10⁻⁹ meters through skin of a patient to a photocell whereby light is converted to current within the patient without a break in the skin of the patient;
a conductor connecting the photocell to the implant whereby the current is applied to the implant.

19. An apparatus in accordance with claim 18 in which the electromagnetic energy is in a wavelength range of 4×10⁻⁷ to 8×10⁻⁷.

20. An apparatus in accordance with claim 18 further comprising means for transmitting signals through the skin from inside the patient to an external apparatus without a break in the skin.

21. An apparatus in accordance with claim 20 further comprising means for using the signals transmitted through the skin to an external apparatus to control the intensity of light transmitted from the external apparatus through the skin to an internal transducer.

22. An apparatus in accordance with claim 20 further comprising means for using the signals transmitted through the skin to an external apparatus to indicate the battery condition of storage batteries in an internal transducer.

23. An apparatus in accordance with claim 18 further comprising:
means for modulating the electromagnetic energy transmitted through the skin of the patient; and
means for using the modulated electromagnetic energy to transmit signals to an internal transducer.

24. An apparatus in accordance with claim 20 further including the step of using the signals transmitted through the skin to indicate the patient's condition.
